(19) United States
(12) Patent Application Publication
(10) Pub. No.: US 2008/0027033 A1
(43) Pub. Date: Jan. 31, 2008

(54) Method and Composition for Treatment of Cutaneous Lesions

(75) Inventors: Igor Gonda, Victoria (AU); Timothy Matthias Morgan, Victoria (AU); Nina Frances Wilkins, Victoria (AU)

Correspondence Address: FOLEY AND LARDNER LLP;SUITE 500, 3000 K STREET NW, WASHINGTON, DC, 20007, US

(73) Assignee: Acrux DDS Pty Ltd

(21) Appl. No.: 10/579,756

(22) PCT Filed: Nov. 19, 2004
(86) PCT No.: PCT/AU04/01609

§ 371 Date: Jan. 29, 2007

Related U.S. Application Data
(60) Provisional application No. 60/523,138, filed on Nov. 19, 2003.

(51) Int. Cl. A61K 31/56; A61K 31/47; A61P 17/02; A61K 31/4745

(52) U.S. Cl. 514/169; 514/292; 514/311

(57) ABSTRACT

A method of treatment or prophylaxis of a cutaneous lesion in an animal the method comprising topically applying to an area of skin of the animal a composition comprising: one or more metal chelators; one or more transforming growth factor modulators; and one or more dermal penetration enhancers.

### FIELD

The present invention relates to a method and composition for the treatment cutaneous lesions, such as a diabetic ulcer or venous ulcer in an animal subject such as a human.

### BACKGROUND

The effective treatment of cutaneous lesions, particularly chronic cutaneous ulceration, has long been a considerable challenge for medical science. Cutaneous lesions may arise as a result of chronic steroid therapy for autoimmune disease or atopic dermatitis, chemotherapy, diabetes, pressure sores and venous disease. These cutaneous open lesions can often persist chronically for extended periods of time. One example of particular concern is the treatment of cutaneous lesions which present as a result of venous diseases. Venous valves control the flow of blood from the superficial veins to the deep veins in a distal to proximal direction. Incompetent valves allow backflow of blood when the muscles of the leg relax, contributing to venous pressures that are higher than normal. This venous hypertension is a major factor in chronic venous insufficiency (CVI).

CVI is a general term which encompasses a number of different changes that can occur in the gaiter area of the leg (lower leg). These changes are due to the longstanding high pressure in the veins which usually occurs because blood flow in the veins is abnormal, but may also occur if veins in the legs become blocked. CVI leads to pooling of blood and fluid in the extremities, causing swelling, mild redness and scaling of the skin which in turn can lead to ulceration. Venous ulcers can take anywhere from months to years to heal, and recurrence is common.

Traditionally, any ulcers that develop are treated with compressive bandages that often contain antibiotic solutions. However, topical antibiotics have been shown to be ineffective in healing leg ulcers and are likely to produce skin sensitisation, thus patients with venous disease of the lower limb are very likely to become allergic to dressing materials. Recurrent ulceration may be surgically treated with skin grafts and repair or bypass of the affected veins. In the past, drug treatment has been of little benefit. However, in recent years a better understanding of the pathological mechanisms underlying skin damage in venous disease has allowed more rational pharmacotherapeutic approaches to be made.

Existing physiologically active agents such as trafermin, becaplermin and tranilast are expensive and their efficacy is limited by their low percutaneous penetration from around and within the lesion site.

It has been shown that the rate and extent of lesion healing in an animal suffering from a cutaneous lesion is significantly enhanced with the use of a chelating agent. (Allhorn, M et al, 2003, J Invest Dermatol, 121(3): 640-646), in particular, venous ulcers which are lesions on the skin of the ankle or lower leg caused by CVI.

Excess metal deposition has been shown to actively perpetuate tissue damage in venous ulcerations. WO 03/002119, dated Jun. 27 2001, discloses a method for the prevention and treatment of lipodermatosclerosis by administering a metal chelating agent. As preferred compounds, aromatic amines, carbonyls, oximate, enolates, phenoxides, catecholates and hydroxylates are mentioned. More specifically, the use of 1,10-phenanthroline is disclosed. The removal of excess metals was considered to prevent or treat the fibrotic symptoms of lipodermatosclerosis and subsequently alleviate chronic inflammation.

Growth factors have been shown to stimulate neurovascularisation. U.S. Pat. No. 6,541,447 discloses a composition for accelerating wound healing in a subject comprising ovalbumin containing a growth factor including transforming growth factor alpha. Transforming growth factor (TGF) modulators, such as oestrogen, have also been shown to increase the rate of wound healing (Ashcroft, G S et al, 1997, Nature Med, 3(11): 1209-1215) and EP0930876.

There is a need for an enhanced composition and method of treatment of cutaneous lesions.

No admission is made that any reference, including any patent or patent document, cited in this specification constitutes prior art. In particular, it will be understood that, unless otherwise stated, reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY

We have found that metal chelators such as 1,10-phenanthroline in combination with TGF modulators such as oestrogen can be administered transdermally to provide effective prevention and/or treatment of cutaneous lesions. The topical use of dermal penetration enhancer, metal chelator and TGF modulator modifies the lesion repair process and enables an increase in the rate and extent of healing.

In a first aspect the present invention provides a method of treatment or prophylaxis of a cutaneous lesion in an animal the method comprising topically applying to an area of skin of the animal a composition comprising:

one or more metal chelators;

one or more transforming growth factor modulators; and

one or more dermal penetration enhancers.

In a second aspect the invention provides a composition for transdermal administration for treatment or prophylaxis of a cutaneous lesion the composition comprising:

one or more metal chelators;

one or more transforming growth factor modulators; and

one or more dermal penetration enhancers.

In a third aspect the invention provides the use of a transforming growth factor modulator in combination with a metal chelator to increase the rate and extent of lesion healing in an animal suffering from a cutaneous lesion by topical application to the gaiter area of the leg.

In a further aspect the invention provides the use of at least one TGF modulator and at least one metal chelator in the manufacture of a transdermal composition for treatment or prophylaxis of cutaneous lesions.

The composition of the invention may contain one or more volatile liquids, such as ethanol or isopropanol.

It is preferred that, after application of the non-occlusive, percutaneous or transdermal drug delivery system, the volatile component of the delivery system evaporates and the area of skin to which the drug delivery system was applied becomes touch-dry. More preferably said area of skin becomes touch-dry within 3 minutes, more preferably within 1 minute. Once the volatile liquid of the non-occlusive drug delivery system has evaporated, driving the mixture of non-volatile dermal penetration enhancer and active agent into the stratum corneum, the outer surface of the skin is then substantially free of active agent and non-volatile dermal penetration enhancer. Normal touching, wearing of clothes, rinsing or even washing of the skin will not, to any significant extent, affect delivery of the drug or displace either the active agent or the non-volatile dermal penetration enhancer, once the volatile liquid has evaporated.

The present invention uses one or more dermal penetration enhancers for enhanced transdermal drug delivery. The invention may use traditional dosage forms such as gels, lotions and patches. Preferably the composition is applied by spraying the composition onto the skin of the patient.

Preferably the composition is applied daily to the gaiter area of the leg (lower leg), directly on to the cutaneous lesion.

In drug delivery compositions according to the present invention one or more other components selected from the group consisting of active agents, co-solvents, surfactants, emulsifiers, antioxidants, preservatives, stabilisers, diluents and mixtures of two or more of said components may be incorporated as is appropriate to the particular route of administration and dosage form. The amount and type of components used should be compatible with the dermal penetration enhancers of this invention as well as with the metal chelating agent and TGF modulator. A co-solvent or other standard adjuvant, such as a surfactant, may be required to maintain the chelating agent in solution or suspension at the desired concentration.

### DETAILED DESCRIPTION

The composition of the present invention preferably contains from about 0.1% to about 10% of a metal chelator, from about 0.1% to about 10% of a TGF modulator, from about 0.1% to about 10% of a dermal penetration enhancer, and optionally from about 45% to about 99.8% of a volatile solvent.

In another preferred form the volatile liquid is ethanol, isopropanol or mixture thereof in the range of about 80 to 98%. More preferably the composition of the invention will comprise from about 1 to 5% of a metal chelator, from about 1 to 5% of a TGF modulator, from about 2 to 8% of the dermal penetration enhancer, from about 45 to 90% ethanol, isopropanol or mixture thereof, 5 to 45% water; and optionally 0.5 to 5% of a thickening agent.

Suitable metal chelating agents include 8-hydroxy quinoline, 8-hyroxy quinoline-5-sulphonic acid, diethyl dithiocarbamate, phenanthroline and it's derivatives, dipicolinate, diphenylthiocarbazone, dithizone, cimetidine, dipicolinic acid, deferiprone, diacerein, clioquinol or pharmaceutically acceptable salts or derivatives of any one of the aforementioned.

More preferably the chelating agent is 1,10-phenanthroline.

Suitable TGF modulators are oestrogens including oestradiol, oestriol, oestrone, ethinyloestradiol, mestranol, stilboestrol, dienoestrol, epioestriol, estropipate, zeranol or pharmaceutically acceptable salts or derivatives of any one of the aforementioned.

More preferably the oestrogen is oestradiol.

The concentration of metal chelator and oestrogen and the dose of composition applied will be sufficient to provide a therapeutic effect having regard to the specific formulation and the area of topical administration.

The performance of the dermal penetration enhancer to deliver a desired chelating agent and TGF modulator such as oestrogen varies with differences in the nature of the dermal penetration enhancer, the oestrogen and the chelator. It is understood that different dermal penetration enhancers may need to be selected to be appropriate for delivery of various metal chelators.

The dermal penetration enhancer may be selected from the classes of enhancers that are lipophilic non-volatile liquids whose vapour pressure is below 10 mm Hg at atmospheric pressure and normal skin temperature of 32 degrees Celsius. Preferably, the dermal penetration enhancer has a molecular weight within the range of 200 to 400 Daltons.

The dermal penetration enhancers may be selected from the group consisting of fatty acids, fatty acid esters, fatty alcohols, glycols and glycol esters, 1,3-dioxolanes and 1,3-dioxanes, macrocyclic ketones containing at least 12 carbon atoms, oxazolidinones and oxazolidinone derivatives, alkyl-2-(N,N-disubstituted amino)-alkanoate esters, (N,N-disubstituted amino)-alkanol alkanoates, sunscreen esters and mixtures thereof. More preferably the dermal penetration enhancer is selected from the list including oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4-decyloxazolidinon-2-one, octyl dimethyl-para-aminobenzoate, octyl para-methoxycinnamate, octyl salicylate and mixtures thereof.

Preferably the class of dermal penetration enhancers are safe skin-tolerant ester sunscreens.

### BRIEF DESCRIPTION OF THE FIGURES

FIG. 1 Graph showing the predicted cumulative amount of 1,10-phenanthroline diffused across skin;

FIG. 2 Graph showing the predicted cumulative amount of estradiol diffused across skin.

The terms “topical” and “transdermal” are used herein in the broadest sense to refer to administration of a drug to the skin surface or mucosal membrane of an animal, including humans, so that the drug passes through the skin tissue. Unless otherwise stated or implied, the terms topical drug delivery and transdermal drug delivery are used interchangeably.

The term “dermal penetration enhancer” is used herein in its broadest sense to refer to an agent which improves the rate of percutaneous transport of active agents into and/or across the skin or use and delivery of active agents to organisms such as animals, whether it be for local application or systemic delivery.

Preferably the animal is a human but the invention also extends to the treatment of non-human animals.

### EXAMPLE 1

Combined transdermal spray composition

| Component | Amount (% w/v) |
|---|---|
| 1,10-phenanthroline | 5.0 |
| Estradiol | 0.5 |
| Octyl salicylate | 5.0 |
| Ethanol 95% | to volume |

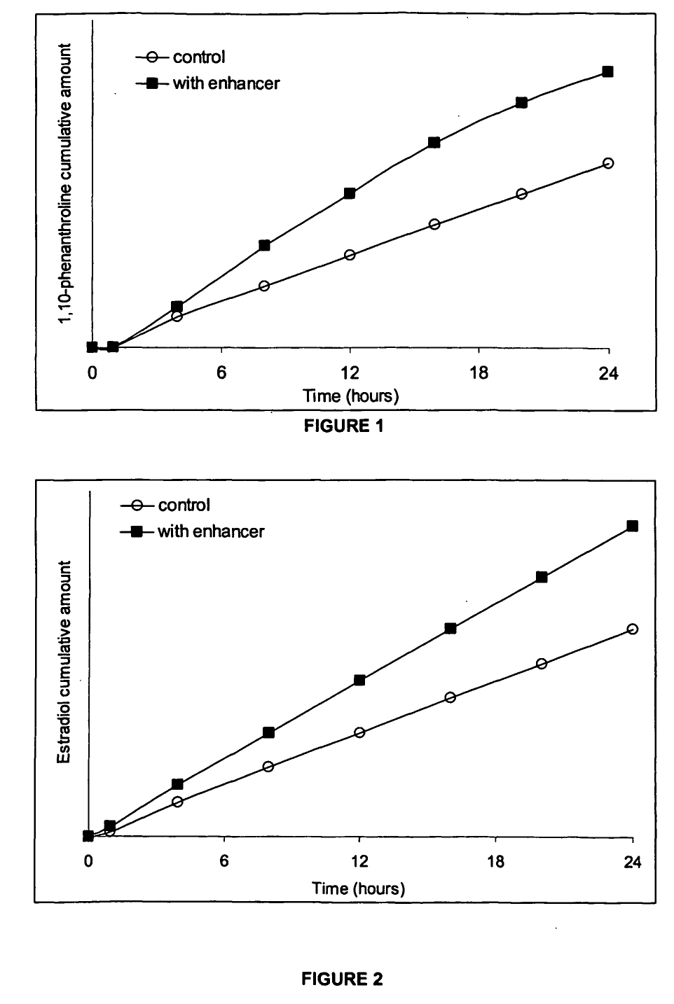

FIGS. 1 and 2 depict the diffusion profile that may be obtained by transdermal administration of 1,10-phenanthroline and estradiol in accordance with the invention. Addition of the octyl salicylate to the transdermal spray formulation causes a significant marked increase in the amount of 1,10-phenanthroline and estradiol diffusing across the skin over 24 hours.

### EXAMPLE 2

Combined transdermal spray composition

| Component | Amount (% w/v) |
|---|---|
| Deferiprone | 2.0 |
| Estradiol | 0.5 |
| Octyl salicylate | 5.0 |
| Alcohol USP (95%) | to volume |

### EXAMPLE 3

Combined transdermal spray composition

| Component | Amount (% w/v) |
|---|---|
| Diacerein | 2.0 |
| Estradiol | 0.5 |
| Padimate-O | 5.0 |
| Alcohol USP (95%) | to volume |

We claim:

1. A method of treatment or prophylaxis of a cutaneous lesion in an animal the method comprising topically applying to an area of skin of the animal a composition comprising:
one or more metal chelators;
one or more transforming growth factor modulators; and
one or more dermal penetration enhancers.

2. A method according to claim 1 wherein animal is suffering CVI and the composition is applied to the gaiter area of the leg of the animal.

3. A method according to claim 1 wherein the composition contains one or more volatile liquids.

4. A method according to claim 3 wherein the one or more volatile liquids are selected from the group consisting of ethanol, isopropanol and mixtures thereof.

5. A method according to claim 1 wherein the composition is in a dosage form selected from the group consisting of gels, lotion patches and aerosol sprays.

6. A method according to claim 1 wherein the composition is applied by spraying the composition onto the skin of the animal.

7. A method according to claim 1 wherein the composition comprises at least one additional component selected from the group consisting of active agents, co-solvents, surfactants, emulsifiers, antioxidants, preservatives, stabilisers, diluents and mixtures of two or more of said components.

8. A method according to claim 1 wherein the composition comprises sufficient co-solvent and/or surfactant to maintain the chelating agent in solution or suspension at the desired concentration.

9. A method according to claim 1 wherein the composition contains from about 0.1% to about 10% of a metal chelator, from about 0.1% to about 10% of a TGF modulator, from about 0.1% to about 10% of a dermal penetration enhancer.

10. A method according to claim 9 wherein the composition comprises from about 45% to about 99.8% of a volatile solvent.

11. A method according to claim 10 wherein the volatile solvent is selected from the group consisting of ethanol, isopropanol and mixture thereof in and amount in the range of about 80 to about 98%.

12. A method according to claim 1 wherein the composition comprises from about 1 to 5% of a metal chelator, from about 1 to 5% of a TGF modulator, from about 2 to 8% of the dermal penetration enhancer, from about 45 to 90% ethanol, isopropanol or mixture thereof and 5 to 45% water.

13. A method according to claim 11 wherein the composition further comprises 0.5 to 5% of a thickening agent.

14. A method according to claim 1 wherein the chelating agent comprises at least one compound selected from the group consisting of 8-hydroxy quinoline, 8-hydroxy quinoline-5-sulphonic acid, diethyl dithiocarbamate, phenanthroline and it's derivatives, dipicolinate, diphenylthiocarbazone, dithizone, cimetidine, dipicolinic acid, deferiprone, diacerein, clioquinol, pharmaceutically acceptable salts thereof and derivatives of the aforementioned.

15. A method according to claim 1 wherein the chelating agent is 1,10-phenanthroline.

16. A method according to claim 1 wherein the composition comprises at least one oestrogen selected from the group consisting of oestradiol, oestriol, oestrone, ethinyloestradiol, mestranol, stilboestrol, dienoestrol, epioestriol, estropipate, zeranol or pharmaceutically acceptable salts or derivatives of any one of the aforementioned.

17. A method according to claim 15 wherein the oestrogen is oestradiol.

18. A method according to claim 1 wherein the one or more dermal penetration enhancers are selected from the group consisting of fatty acids, fatty acid esters, fatty alcohols, glycols and glycol esters, 1,3-dioxolanes and 1,3-dioxanes, macrocyclic ketones containing at least 12 carbon atoms, oxazolidinones and oxazolidinone derivatives, alkyl-2-(N,N-disubstituted amino)-alkanoate esters, (N,N-disubstituted amino)-alkanol alkanoates, sunscreen esters and mixtures thereof.

19. A method according to claim 1 wherein the one or more dermal penetration enhancers are selected from the group consisting of oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4-decyloxazolidinon-2-one, octyl dimethyl-para-aminobenzoate, octyl para-methoxycinnamate, octyl salicylate and mixtures thereof.

20. A method according to claim 1 wherein the one or more dermal penetration enhancers are selected from safe skin-tolerant ester sunscreens.

21. A method according to claim 1 wherein the one or more penetration enhancers are selected from the group consisting of octyl dimethyl-para-aminobenzoate, octyl para-methoxycinnamate and octyl salicylate.

22. A composition for transdermal administration for treatment or prophylaxis of cutaneous lesions in animals the composition comprising:
one or more metal chelators;
one or more transforming growth factor modulators; and
one or more dermal penetration enhancers.

23. A composition according to claim 22 wherein the composition further comprises one or more volatile liquids.

24. A composition according to claim 22 wherein the one or more volatile liquids are selected from the group consisting of ethanol, isopropanol and mixtures thereof.

25. A composition according to claim 22 wherein the composition is in a dosage form selected from the group consisting of gels, lotion patches and aerosol sprays.

26. A composition according to claim 22 wherein the composition comprises at least one additional component selected from the group consisting of active agents, co-solvents, surfactants, emulsifiers, antioxidants, preservatives, stabilisers, diluents and mixtures of two or more of said components.

27. A composition according to claim 22 wherein the composition comprises sufficient of at least one of a co-solvent and surfactant to maintain the chelating agent in solution or suspension.

28. A composition according to claim 22 wherein the composition contains from about 0.1% to about 10% of the metal chelator, from about 0.1% to about 10% of the TGF modulator and from about 0.1% to about 10% of the dermal penetration enhancer.

29. A composition according to claim 28 wherein the composition comprises from about 45% to about 99.8% of a volatile solvent.

30. A composition according to claim 28 wherein the volatile solvent is selected from the group consisting of ethanol, isopropanol and mixture thereof in and amount in the range of about 80 to about 98%.

31. A composition according to claim 28 wherein the composition comprises from about 1 to 5% of a metal chelator, from about 1 to 5% of a TGF modulator, from about 2 to 8% of the dermal penetration enhancer, from about 45 to 90% ethanol, isopropanol or mixture thereof and 5 to 45% water.

32. A composition according to claim 28 wherein the composition further comprises 0.5 to 5% of a thickening agent.

33. A composition according to claim 28 wherein the chelating agent comprises at least one compound selected from the group consisting of 8-hydroxy quinoline, 8-hydroxy quinoline-5-sulphonic acid, diethyl dithiocarbamate, phenanthroline and it's derivatives, dipicolinate, diphenylthiocarbazone, dithizone, cimetidine, dipicolinic acid, deferiprone, diacerein, clioquinol, pharmaceutically acceptable salts thereof and derivatives of the aforementioned.

34. A composition according to claim 28 wherein the chelating agent is 1,10-phenanthroline.

35. A composition according to claim 28 wherein the composition comprises at least one oestrogen selected from the group consisting of oestradiol, oestriol, oestrone, ethinyloestradiol, mestranol, stilboestrol, dienoestrol, epioestriol, estropipate, zeranol or pharmaceutically acceptable salts or derivatives of any one of the aforementioned.

36. A composition according to claim 28 wherein the oestrogen is oestradiol.

37. A composition according to claim 28 wherein the one or more dermal penetration enhancers are selected from the group consisting of fatty acids, fatty acid esters, fatty alcohols, glycols and glycol esters, 1,3-dioxolanes and 1,3-dioxanes, macrocyclic ketones containing at least 12 carbon atoms, oxazolidinones and oxazolidinone derivatives, alkyl-2-(N,N-disubstituted amino)-alkanoate esters, (N,N-disubstituted amino)-alkanol alkanoates, sunscreen esters and mixtures thereof.

38. A composition according to claim 28 wherein the one or more dermal penetration enhancers are selected from the group consisting of oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4-decyloxazolidinon-2-one, octyl dimethyl-para-aminobenzoate, octyl para-methoxycinnamate, octyl salicylate and mixtures thereof.

39. A composition according to claim 28 wherein the one or more dermal penetration enhancers are selected from safe skin-tolerant ester sunscreens.

40. A composition according to claim 28 wherein the one or more penetration enhancers are selected from the group consisting of octyl dimethyl-para-aminobenzoate, octyl para-methoxycinnamate and octyl salicylate.
